(19) United States
(12) Patent Application Publication
(10) Pub. No.: US 2026/0354837 A1
(43) Pub. Date: Nov. 12, 2026

(54) DEVICE FOR ALIGNING A TIBIAL CUTTING BLOCK, AND METHOD FOR CONFIGURING THE DEVICE

(71) Applicant: Aesculap AG, Tuttlingen (DE)

(72) Inventors: Franz-Peter Firmbach, Emmingen-Liptingen (DE); Stephanie Stoll, Villingendorf (DE)

(21) Appl. No.: 19/716,638

(22) Filed: Jun. 23, 2026

Related U.S. Application Data
(63) Continuation of application No. PCT/EP2025/050030, filed on Jan. 2, 2025.

(30) Foreign Application Priority Data
Jan. 5, 2024 (DE) 10 2024 100 271.9

(51) Int. Cl. A61B 17/15

(52) U.S. Cl. CPC A61B 17/157

(57) ABSTRACT

A device for kinematically aligning a tibial cutting block includes a first correction device and a second correction device. The first correction device is configured for correcting a flexion angle. The second correction device is configured for correcting a tibial cutting height. The first correction device and the second correction device include a guide for a rod for aligning the tibial cutting block. The guide is rotatable in order to correct the flexion angle. The rod is linearly movable with respect to the guide in order to correct the tibial cutting height. The device can be part of an instrument set, and configured in a method in which at least one alignment rod is arranged relative to a receptacle.

### CROSS-REFERENCE TO RELATED APPLICATIONS

This application is a continuation of International Application No. PCT/EP2025/050030, filed on Jan. 2, 2025, under 35 U.S.C. § 365, and claims priority to German Application No. 10 2024 100 271.9, filed on Jan. 5, 2024, under 35 U.S.C. § 119. The contents of International Application No. PCT/EP2025/050030 and German Application No. 10 2024 100 271.9 are incorporated by reference herein in their entireties.

### FIELD

The present disclosure relates to a device for kinematically aligning a tibial cutting block and to a method for configuring the device.

### BACKGROUND

U.S. Pat. No. 10,709,458 B1 discloses a surgical tool and a method for kinematic alignment in total knee arthroplasty, which enables kinematic alignment by adjusting a tibial slope, wherein a tibial cutting height changes with the adjusted tibial slope.

### SUMMARY

The device and method for kinematically aligning a tibial cutting block offer an easy-to-use way to transfer the tibial cutting height when transferring a cut from the femur to the tibia, to compensate for a change in tibial cutting height resulting from forced flexion of the knee, and to set a native tibial slope according to a preoperative plan.

The device comprises a first correction device and a second correction device, wherein the first correction device is configured to correct a flexion angle, wherein the second correction device is configured to correct a tibial cutting height, wherein the first correction device and the second correction device comprise a guide for a rod for aligning the tibial cutting block, wherein the guide for correcting the flexion angle is rotatable, and wherein the rod for correcting the tibial cutting height is linearly movable with respect to the guide.

For example, the first correction device and the second correction device have means for adjusting a correction, adapted to the correction of the flexion angle, of the tibial cutting height.

The means for adjusting the correction, adapted to the correction of the flexion angle, of the tibial cutting height comprise, for example, a first scale and a second scale, wherein the correction of the flexion angle is adjustable on the first scale, wherein the correction of the tibial cutting height is adjustable on the second scale, and wherein the first scale and the second scale uniquely identify corrections of the flexion angle and the tibial cutting height adapted to each other, in particular with the same scale value, preferably the same angle specification.

The device comprises, for example, an alignment rod for alignment on an anterior tibial edge and at least one receptacle for the alignment rod, wherein the alignment rod and the receptacle comprise means for adjusting an angle at which the alignment rod extends from the receptacle.

The device comprises, for example, two receptacles that are arranged at different distances from a posterior side of the second fastening part.

The means for adjusting the angle include, for example, an angle at which the receptacle extends with respect to a mechanical axis and at least one angle in the alignment rod.

The first correction device comprises, for example, a fastening part for fastening the device to the femur or a femur trial implant, wherein the guide is rotatable with respect to the fastening part for adjusting the correction of the flexion angle.

The rod has, in particular on its posterior side, a toothed rack extending between a proximal and a distal end of the rod, wherein the guide has a gearwheel, and wherein the gearwheel and the rod are arranged in the guide such that the gearwheel engages with the toothed rack.

An instrument set comprises the device, wherein the device comprises at least one receptacle for an alignment rod and the instrument set comprises alignment rods with different angles or an alignment rod with various angle adjustment options.

The method for configuring the device provides that alignment rods with different angles, or an alignment rod with various angle adjustment options, be provided, wherein an alignment rod is arranged such that the alignment rod extends from the recess at a selected angle.

### BRIEF DESCRIPTION OF THE DRAWINGS

Further advantageous embodiments can be found in the following description and drawings. In the drawings:

FIG. 1 is a schematic illustration of a side view of a device for kinematically aligning a tibial cutting block;

FIG. 2 is a schematic illustration of a side view of a first part of the device;

FIG. 3 is a schematic illustration of a side view of a second part of the device;

FIG. 4 is a schematic illustration of a side view of a third part of the device;

FIG. 5 is a schematic illustration of a side view of a fourth part of the device;

FIG. 6 is a schematic illustration of a fifth part of the device; and

FIG. 7 is a schematic illustration of a section through part of the device.

### DETAILED DESCRIPTION

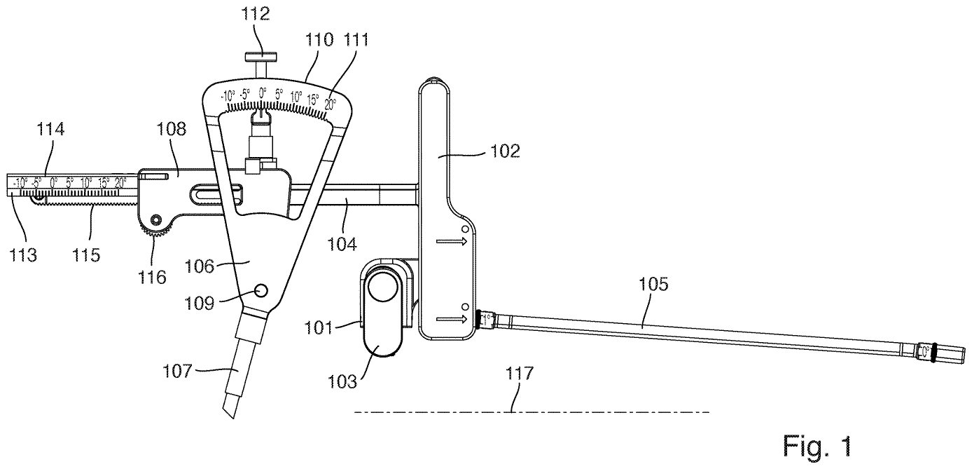

FIG. 1 shows a schematic illustration of a side view of a device 100 for kinematically aligning a tibial cutting block.

The tibial cutting block is not shown in FIG. 1.

The device 100 comprises a first fastening part 101. In the example, the first fastening part 101 comprises a coupling device 103 for fastening the tibial cutting block to the first fastening part 101.

The device 100 comprises a second fastening part 102.

The first fastening part 101 is designed to fasten the first fastening part 101 to the second fastening part 102.

The second fastening part 102 is designed to fasten the second fastening part 102 to the first fastening part 101.

In the example, the first fastening part 101 and the second fastening part 102 are adjustable with respect to each other anteriorly and posteriorly.

It may be provided that the first fastening part 101 and the second fastening part 102 be fixed immovably with respect to each other anteriorly and posteriorly.

The second fastening part 102 has a rod 104 for correcting the tibial cutting height. The rod 105 is arranged on the proximal side of the second fastening part 102. The rod 104 extends from the second fastening part 102 proximally to a proximal end of the device 100. In this example, the rod 104 and the second fastening part 102 are one piece.

The second fastening part 102 is designed for fastening an alignment rod 105 to the second fastening part 102.

In the example, the device 100 comprises the alignment rod 105. The alignment rod 105 is designed to align the device 100 with an anterior tibial edge.

The alignment rod 105 is arranged on the distal side of the second fastening part 102 at the distal end of the device 100. The alignment rod 105 extends distally from the second fastening part 102 to the distal end of the device 100.

The device 100 has a third fastening part 106.

The second fastening part 102 is designed to fasten the second fastening part 102 to the third fastening part 106.

The third fastening part 106 is designed to fasten the third fastening part 106 to the second fastening part 102.

In the example, the second fastening part 102 and the third fastening part 106 are fastened to each other in a proximally and distally adjustable manner.

The third fastening part 106 has a posterior end 107 on which the device 100 can be arranged on the femur or on a femur trial implant.

For example, the end 107 is designed to position the device 100 on two posterior-distal bores made in the femur. The end 107 comprises, for example, a posteriorly-extending positioning dowel for each bore, which is designed to be inserted into the corresponding bore.

For example, a locking mechanism is provided on the end 107 and is designed to arrange the end 107 on the positioning dowels. The positioning dowels, the bore and the locking mechanism are not shown in FIG. 1.

The locking mechanism comprises, for example, a wing screw for each positioning dowel, with which the respective positioning dowels can be fixed in the corresponding bores.

The device 100 has a guide 108. The rod 104 is linearly movable with respect to the guide 108. The guide 108 is designed to guide the rod 104 laterally, medially, anteriorly and posteriorly in a proximal or distal movement of the rod 104 with respect to the third fastening part 106 to correct the tibial cutting height.

The guide 108 is rotatably mounted on the third fastening part 106 about an axis of rotation 109, for correcting the flexion angle. The axis of rotation 109 extends from a lateral side of the third fastening part 106 to a medial side of the third fastening part 106.

The third fastening part 106 has a bracket 110 at the front. The bracket 110 has a first scale 111 for correcting the flexion angle. In the example, the first scale 111 is an angular measure scale. The first scale 111 indicates the correction of the flexion angle between −10° and +20° in one-degree increments around a zero position of the first scale 111 marked 0°. In this example, the zero position of the first scale 111 corresponds to a correction of the flexion angle by 0°, i.e., no correction of the flexion angle.

In the example, the device 100 is designed to compensate for a bending deficit of up to 20°. Other angular ranges are also possible-for example, up to a bending deficit between 10° and 15°, especially 10° or 15°. This eliminates the need to loosen the ligaments in the knee to allow the leg to extend further.

In the example, the guide 108 has a device 112.

In the example, the device 112 is designed to display the correction of the flexion angle on the bracket 110.

In the example, the device 112 is designed to fix the bracket 110 to set an adjusted correction of the flexion angle. For example, the device 112 is designed to clamp the bracket 110. In the example, the device 112 is configured to release the bracket 110 for adjusting a correction of the flexion angle for a rotation of the guide 108 with respect to the third fastening part 106. For example, the device 112 comprises a screw clamp designed to clamp or release the bracket 110.

This fixes the guide 108 relative to the third fastening part 106 at a set rotation angle, i.e., the desired correction of the flexion angle.

The device 100 has a first profile 113 with a second scale 114. The second scale 114 in the example is a template scale for setting the tibial cutting height.

The profile 113 is arranged on the guide 108. In this example, the profile 113 is arranged on a proximal side of the guide 108. In this example, the profile 113 extends proximally from the third fastening part 106.

In the example, the rod 104 is arranged to be movable in the proximal and distal direction with respect to the profile 113. In the example, the profile 113 is designed to guide the rod 104 in a movement of the rod in proximal and distal directions in medial and lateral directions.

In the example, the profile 113 is arranged at the proximal end of the device 100. In the example, the profile 113 is arranged at the proximal end of the rod 104. The second scale 114 is designed to indicate the correction of the tibial cutting height. The second scale 114 extends from a proximal side of the profile 113 to a distal side of the profile 113.

The first scale 111 and the second scale 114 are coordinated. The example shows a correlation between the first scale (111) and the second scale (114).

In the example, the first scale 111 indicates the correction of the flexion angle between −10° and +20° in one-degree increments around a zero position of the first scale 111 marked 0°. The zero position of the second scale 114 corresponds to the correction of the tibial cutting height, which is provided in the zero position of the first scale 111 with a correction of the flexion angle by 0°, i.e., no correction of the flexion angle. In the example, it is assumed that the tibial cutting height in the zero position of the second scale 114 is a prespecified height, e.g., 10 mm. The tibial cutting height associated with the zero position can be more or less than 10 mm.

The first scale 111 and the second scale 114 are coordinated in such a way that the tibial cutting height is the specified height, e.g., 10 mm, when the same angle is set on the first scale 111 and the second scale 114.

In the example, the rod 104 is movable with respect to the second scale 114. In the example, rod 104 has a toothed rail 115. In the example, the guide 108 has a gearwheel 116. In this example, the toothed rail 115 and the gearwheel 116 mesh together. The gearwheel 116 and the toothed rail 115 are designed to enable the distal and proximal movement of the rod 104 in the guide 108 in the region of the second scale 114. This means that the rod 104 can be extended or retracted according to the adjusted correction of the flexion angle, so that the tibial cutting height is the specified height, e.g., 10 mm, when the rod 104 is set correctly.

When the device 100 is used, the device 100 is arranged such that the alignment rod 105 extends parallel or substantially parallel to the anterior edge of the tibia.

The first fastening part 101, the second fastening part 102, the end 107, the rod 104 and the alignment rod 105 are arranged at angles to each other, producing as a result the height as the tibial cutting height.

In the example, it is provided that the rod 104 extend parallel or substantially parallel to the mechanical axis 117 when the device 100 is used.

The guide 108, which is rotatable with respect to the third fastening part 106, and the first scale 111 constitute parts of a first correction device designed to correct the flexion angle.

The rod 104, movable in the guide 108, and the second scale 114 constitute parts of a second correction device designed to correct the tibial cutting height.

The first scale 111 and the second scale 114 constitute means for adjusting a correction, adapted to the correction of the flexion angle, of the tibial cutting height.

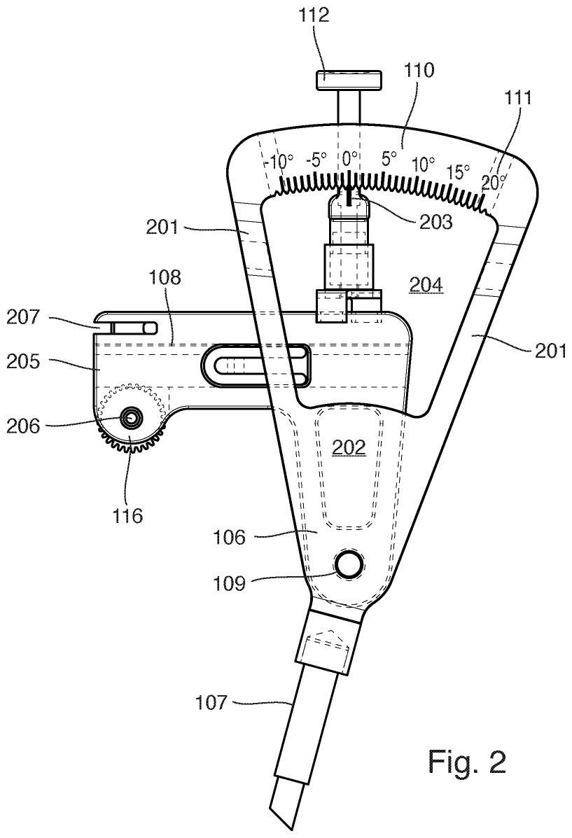

FIG. 2 shows a side view of the third fastening part 106 and the guide 108.

In the example, the bracket 110 is connected to a body 202 of the fastening part 106 by two webs 201. In the example, the device 112 has a pointer 203 which is designed to indicate the correction of the flexion angle on the first scale 111. In the example, the pointer 203 is arranged in a recess 204 in the third fastening part 106. In the example, the recess 204 is enclosed by the body 202, the webs 201 and the bracket 110.

In the example, the axis of rotation 109 is arranged between the recess 204 and the end 107 in the body 202.

The guide 108 comprises a channel 205 which is designed to accommodate the rod 104. In the example, the gearwheel 116 is rotatably arranged in the guide 108 about an axis 206 extending from the lateral to the medial side of the guide 108. In the example, the axis 206 is arranged on the proximal posterior side of the guide 108 next to the channel 205. The gearwheel 116 is designed to engage with the teeth of the toothed rack 115 when the toothed rack 115 is arranged in the channel 205.

The guide 108 has on its proximal side a groove 207 for attaching the profile 113 to the guide 108.

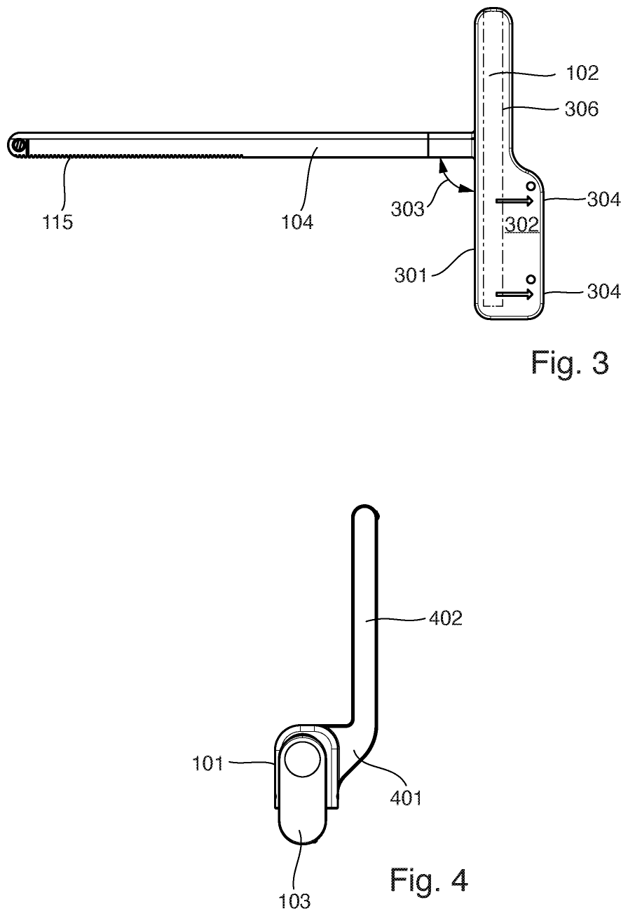

FIG. 3 shows a schematic side view of the second fastening part 102. In the example, the rod 104 is arranged at an angle 303, in particular a right angle, with respect to a proximal edge 301 of a body 302 of the second fastening part 102.

The second fastening part 102 has, in the example, two receptacles 304 for the alignment rod 105, which are spaced apart from each other on the distal side of the body 302. In the example, one of the receptacles 304 is arranged on the posterior side of the body 302, wherein the other receptacle 304 is arranged further away from the posterior side of the body 302. This allows setting different distances of the alignment rod 105 from the anterior edge of the tibia.

The second fastening part 102 has a receptacle 306 in the body 302, which extends in the body 302 from the anterior to the posterior side of the body 302. The receptacle 306 is designed to receive a rod arranged on the first fastening part 101.

FIG. 4 schematically shows a side view of the first fastening part 101 with the coupling device 103 for fastening the tibial cutting block to the first fastening part 101. The first fastening part 101 comprises a body 401 which comprises the coupling device 103. The body 401 has the rod 402 for connecting the first fastening part 101 with the second fastening part 102. In this example, the rod 402 extends anteriorly from the body 401 on an anterior side of the body 401.

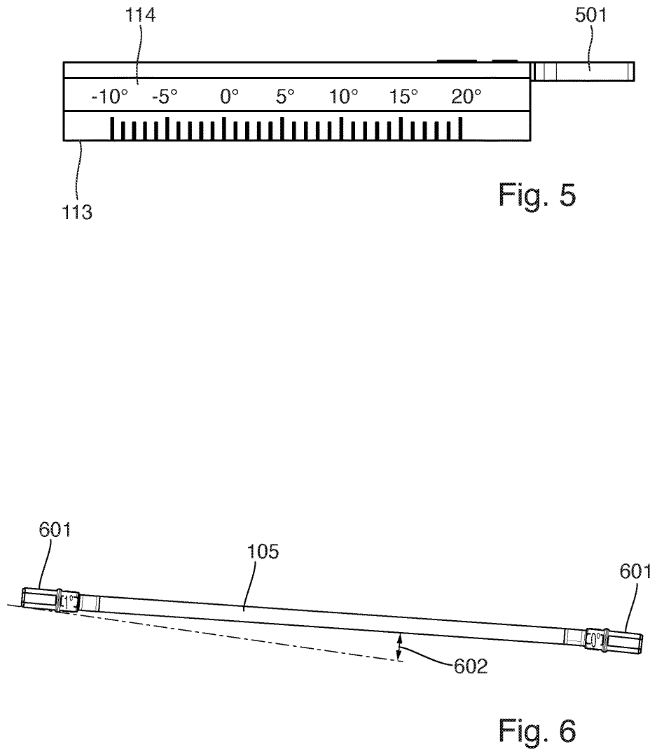

FIG. 5 shows a side view of the profile 113 with the second scale 114.

The profile 113 has a fastening element 501 on its distal side for fastening the profile 113 to the guide 108. The fastening element 501 comprises, for example, a spring that can be inserted into the groove 207 in the guide 108.

FIG. 6 shows a schematic side view of the alignment rod 105. The alignment rod 105 has two end faces 601 which are designed to be received by the respective receptacles 304 in the second fastening part 102.

The alignment rod 105 has at least one angle 602 on at least one of its end faces 601. The angle 602 changes the direction in which the alignment rod 105 extends from the given receptacle 304.

The alignment rod 105 can be arranged so that the alignment rod 105 extends to the second fastening part 102 at an angle selectable from the angles provided on the alignment rod 105.

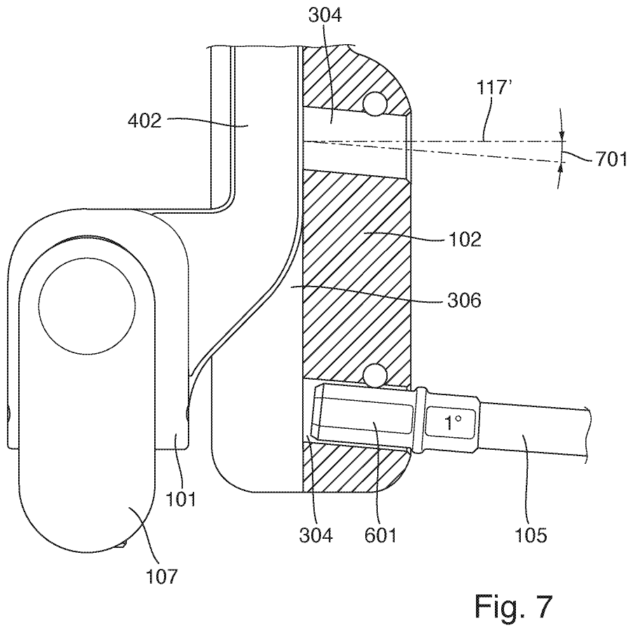

FIG. 7 schematically shows a section through the second fastening part 102, the receptacle 306 for the rod 402 and the receptacles 304 for the alignment rod 105.

With an alignment rod 105, four angles 0°, 1°, 2°, 3°, 4°, for example, can be set.

The receptacle for the alignment rod 105 is, for example, arranged at an angle 701, e.g., of 5°, to the mechanical axis. A parallel 117′ to the mechanical axis is shown in FIG. 7.

In the example, the device 100 is designed for a typical angle of 3° between the mechanical axis and the anterior edge of the tibia.

The angle of 5° comprises an angle of −2° as an offset to the angle of 3°, which represents the typical angle between the mechanical axis and the anterior tibial edge. The offset of −2° is designed so that the entire correction can be carried out with only one alignment rod 105. The offset of −2° is compensated for by the angle designated with the first value 2 in the following table. The second value in the table is an indication on the alignment rod 105, which specifies a correction of the typical angle (3°) between the mechanical axis and the anterior tibial edge in magnitude and sign. The result in the table represents the total correction of the typical angle (3°) by the alignment rod 105. In the example, the second value, 0, reverses the correction of the offset of −2°:

2−2=0

2−1=1

2+0=2

2+1=3

2+2=4

For example, various alignment rods 105 with different angles or an alignment rod 105 with different angle adjustment options are provided.

To set up the device 100, the device 100 is assembled. The alignment rod 105 is arranged such that the alignment rod 105 extends from the recess 304 at a selected angle.

In this example, the correction of the flexion angle and the correction of the tibial cutting height are carried out after the device 100 is positioned on the femur or on the femur trial implant.

1. A device for kinematically aligning a tibial cutting block, the device comprising:
a first correction device configured for correcting a flexion angle; and
a second correction device configured for correcting a tibial cutting height,
the first correction device and the second correction device comprising a guide for a rod for aligning the tibial cutting block,
the guide being rotatable to correct the flexion angle, and
the rod being linearly movable with respect to the guide to correct the tibial cutting height.

2. The device according to claim 1, wherein the first correction device and the second correction device comprise indicia, the indicia configured to adjust a correction, adapted to the correction of the flexion angle, of the tibial cutting height.

3. The device according to claim 2, wherein the indicia comprise a first scale and a second scale.

4. The device according to claim 3, wherein the correction of the flexion angle is adjustable on the first scale.

5. The device according to claim 4, wherein the correction of the tibial cutting height is adjustable on the second scale.

6. The device according to claim 5, wherein the first scale and the second scale uniquely identify corrections of the flexion angle and the tibial cutting height adapted to each other.

7. The device according to claim 6, wherein the first scale and the second scale uniquely identify corrections of the flexion angle and the tibial cutting height adapted to each other, with a common scale value.

8. The device according to claim 7, wherein the first scale and the second scale uniquely identify corrections of the flexion angle and the tibial cutting height adapted to each other, with a common angle specification.

9. The device according to claim 1, further comprising:
an alignment rod for alignment on an anterior tibial edge; and
at least one receptacle for the alignment rod,
the alignment rod and the at least one receptacle comprising means for adjusting an angle at which the alignment rod extends from the at least one receptacle.

10. The device according to claim 9, wherein the means for adjusting the angle comprise an angle at which the at least one receptacle extends with respect to a mechanical axis and comprise at least one angle in the alignment rod.

11. The device according to claim 9, wherein the first correction device comprises a first fastening part for fastening the device to a femur or a femur trial implant.

12. The device according to claim 11, wherein the guide is rotatable with respect to the first fastening part for adjusting a correction of the flexion angle.

13. The device according to claim 11, further comprising a second fastening part, wherein the at least one receptacle comprises two receptacles that are arranged at different distances from a posterior side of the second fastening part.

14. The device according to claim 1, wherein the rod comprises a proximal end, a distal end, and a toothed rack extending between the proximal end and the distal end.

15. The device according to claim 14, wherein the guide has a gearwheel, and wherein the gearwheel and the rod are arranged in the guide in such a manner that the gearwheel engages in the toothed rack.

16. An instrument set comprising:
the device according to claim 1; and
at least one alignment rod,
the device comprising at least one receptacle for the at least one alignment rod.

17. The instrument set according to claim 16, wherein:
the at least one alignment rod comprises a plurality of alignment rods, and
each of the plurality of alignment rods has a different angle.

18. The instrument set according to claim 16, wherein:
the at least one alignment rod consists of a single alignment rod with various angle adjustment options.

19. A method for configuring the device according to claim 1, the method comprising the steps of:
providing at least one alignment rod;
arranging the at least one alignment rod such that the at least one alignment rod extends from at least one receptacle on the device at a selected angle.

20. The method according to claim 19, wherein the at least one alignment rod comprises either:
a plurality of alignment rods with different angles, or
a single alignment rod with various angle adjustment options.
